(19) United States
(12) Patent Application Publication
(10) Pub. No.: US 2015/0233885 A1
(43) Pub. Date: Aug. 20, 2015

(54) SORPTION-BASED SENSING SYSTEM

(71) Applicants: David Glynn Thomas, Perth (AU); Christopher John Kalli, Perth (AU)

(72) Inventors: David Glynn Thomas, Perth (AU); Christopher John Kalli, Perth (AU)

(73) Assignee: CHEVRON U.S.A. INC., San Ramon, CA (US)

(21) Appl. No.: 14/180,530

(22) Filed: Feb. 14, 2014

(51) Int. Cl. G01N 33/18; G01N 33/28; G01N 21/77

(52) U.S. Cl. CPC G01N 33/1886; G01N 21/7703; G01N 33/2847; G01N 33/1833; G01N 2021/7736; G01N 2021/7723; G01N 2201/088

(57) ABSTRACT

The present invention relates to a sorption-based sensing system for sensing multiple selected species in a fluid. In one aspect, the sensing system comprises an optical conduit for guiding light from an input end to an output end, a detector for detecting at least one feature of the light at the output end associated with the optical characteristic, and an analyzer for determining at least one attribute of at least one of the multiple selected species in the fluid based on the detected feature. The optical conduit includes a sorptive portion having a set of different sorption properties associated with the multiple selected species. The sorptive portion is adapted to be positioned in the fluid to reversibly sorb at least one of the multiple selected species to vary an optical characteristic of the sorptive portion. In another aspect, there is provided a corresponding method for operating the sensing system.

### FIELD OF THE INVENTION

The present invention relates to a sorption-based sensing system.

### BACKGROUND OF THE INVENTION

In the fields of flow measurement, flow control and environmental monitoring, it is often necessary to employ fluid sensors for sensing various characteristics, such as the presence, concentration and level, of selected species in fluids. Considerations for fluid sensors include cost of manufacture, robustness to hostile environments and accuracy of the sensor. A potential application for fluid sensors may be for real time and continuous monitoring of trace materials in fluid-carrying apparatuses such as reservoirs, storage tanks, pipelines and flow streams. These fluid-carrying apparatuses may also be remote and unmanned. It would be beneficial to provide sensors with the above considerations in mind, or at least provide an alternative sensor.

### SUMMARY OF THE INVENTION

According to a first aspect of the invention there is provided a sensing system for sensing multiple selected species in a fluid, the sensing system including:

an optical conduit for guiding light from an input end to an output end, the optical conduit including a sorptive portion having a set of different sorption properties associated with the multiple selected species, the sorptive portion adapted to be positioned in the fluid to reversibly sorb at least one of the multiple selected species to vary an optical characteristic of the sorptive portion;

a detector for detecting at least one feature of the light at the output end associated with the optical characteristic; and

an analyzer for determining at least one attribute of at least one of the multiple selected species in the fluid based on the detected feature.

The sorptive portion may include multiple sorptive elements each exhibiting a subset of the set of different sorption properties. The multiple sorptive elements may each be adapted to sorb a different one or more of the multiple selected species.

The multiple sorptive elements may be multiple sorptive sections of an optical fiber. Alternatively the multiple sorptive elements may be multiple sorptive sections in respective multiple optical fibers.

At least one of the multiple sorptive elements may include a reactive component and a host component that co-operate to provide a desired sorption property of the sorptive element.

The optical characteristic of the sorptive portion may include light confinement characteristic responsive to sorption of one or more of the multiple selected species. In this instance, the at least one feature of the light detected may include optical power.

Alternatively or additionally the optical characteristic of the sorptive portion may include spectroscopic characteristic responsive to sorption of one or more of the multiple selected species. In this instance, the at least one feature detected may include spectral information.

Each of the different sorption properties may be selected from a group consisting of: an absorption property, an adsorption property and an ion-exchange property.

Each sorptive element may include a sorptive outer layer. The sorptive outer layer may include an absorptive cladding layer of an optical fiber. Alternatively or additionally, the sorptive outer layer may include an adsorptive coating layer of an optical fiber.

The optical conduit may include a non-sorptive element for calibration.

The input end and output end may be opposite ends of the optical conduit. Alternatively the input end and output end may be the same end of the optical conduit.

The light source may include a pulsed light source. The pulsed light source may include a pulsed laser.

The light source may include a multi-wavelength light source.

According to a second aspect of the invention there is provided a method for operating a sensing system for sensing multiple selected species in a fluid, the method including the steps of:

providing light to be guided in an optical conduit from an input end to an output end, the optical conduit including a sorptive portion having a set of different sorption properties associated with the multiple selected species, the sorptive portion adapted to be positioned in the fluid to reversibly sorb at least one of the multiple selected species to vary an optical characteristic of the sorptive portion;

detecting at least one feature of the light at the output end associated with the optical characteristic; and

determining at least one attribute of at least one of the multiple selected species in the fluid based on the detected feature.

The step of determining at least one attribute may include determining the presence and/or concentration of the plurality of selected species. The step of determining the presence and/or concentration of the plurality of selected species may include performing a statistical analysis of the detected features. The step of performing a statistical analysis may include performing a principal component analysis.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIGS. 1A and 1B illustrate schematically two configurations of a sensing system.

FIG. 1C illustrates schematically an example of the analyzer illustrated in FIGS. 1A and 1B.

FIG. 2A illustrates schematically an arrangement of the optical conduit illustrated in FIGS. 1A and 1B.

FIG. 2B illustrates schematically another arrangement of the optical conduit illustrated in FIGS. 1A and 1B.

FIG. 2C illustrates schematically yet another arrangement of the optical conduit illustrated in FIGS. 1A and 1B.

FIG. 3A illustrates detected average pulse power over time for the arrangement of the optical conduit illustrated in FIGS. 2A and 2B in the absence of any of the selected species.

FIG. 3B to 3D illustrate detected average pulse power over time for the arrangement of the optical conduit illustrated in FIGS. 2A and 2B in the separate presence of three selected species.

FIG. 3E illustrates detected average pulse power over time for the arrangement of the optical conduit illustrated in FIGS. 2A and 2B in the simultaneous presence of three selected species.

FIG. 4A illustrates detected spectral density versus wavelength for the arrangement of the optical conduit illustrated in FIG. 2C in the absence of any of the selected species.

FIGS. 4B and 4C illustrate detected spectral density versus wavelength for the arrangement of the optical conduit illustrated in FIG. 2C in the separate presence of two selected species.

FIG. 4D illustrates detected spectral density versus wavelength for the arrangement of the optical conduit illustrated in FIG. 2C in the simultaneous presence of two selected species.

FIG. 5 illustrates schematically a method for operating a sensing system.

### DETAILED DESCRIPTION OF EMBODIMENTS

Sensing System

Described herein is a sorption-based sensing system and a method of operating the sensing system. Embodiments of the sensing system facilitate sensing of one or more of multiple selected species in a fluid, and in some cases, differentiating between two or more of the multiple selected species in the fluid. The described sensing system relies on detecting variation in optical characteristics of an optical conduit due to sorption of selected species by a sorptive portion of the optical conduit. Because the sorptive portion is adapted to exhibit a set of different sorption properties with respect to the multiple selected species, the variation in the optical characteristics of the optical conduit contains information indicating the attributes, such as the presence and concentration, of any one or more of the multiple species in the fluid. The variation in the optical characteristics may be ascertained by probing the sorptive portion with the light from a light source.

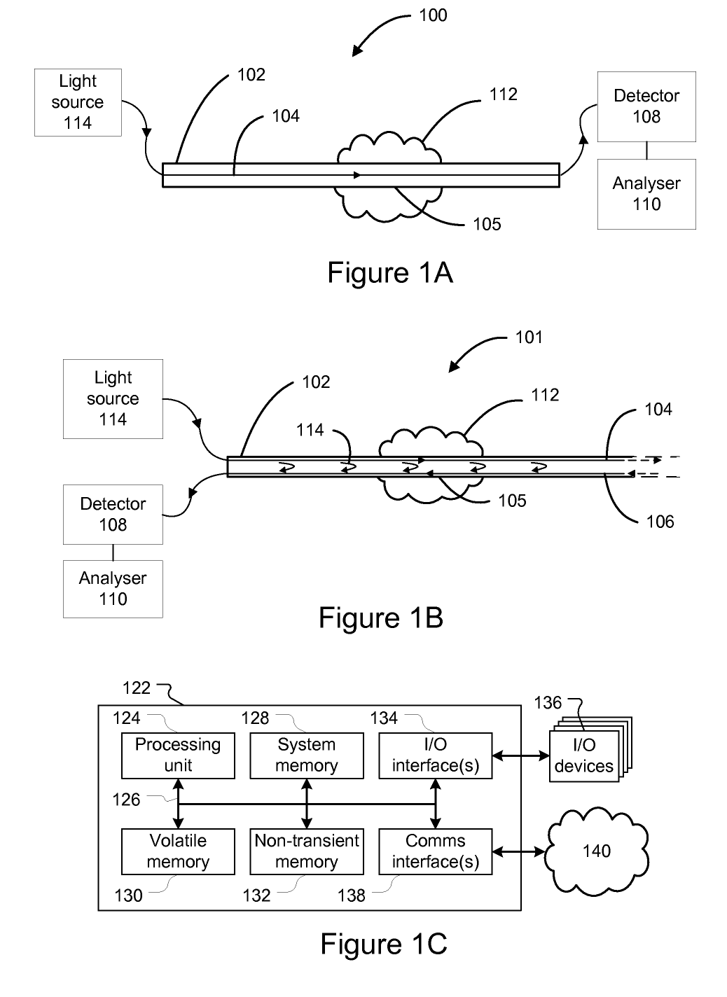

FIG. 1A illustrates a configuration of a sensing system 100. Sensing system 100 includes an optical conduit 102 having two opposite ends, one proximal to a light source 114 and the other distal to the light source 114. Light is received at the proximal end and propagated in a forward direction 104 along optical conduit 102 to the distal end. Optical conduit 102 includes a sorptive portion 105 for immersion or other positioning in a fluid 112. Sensing system 100 also includes a detector 108 for detecting at least one feature of the light provided at the distal end and an analyzer 110 for determining at least one attribute of at least one of the multiple selected species in a fluid 112 based on the detected feature.

Light source 114 may include a laser, such as a pulsed laser. Light source 114 may also include a multi-wavelength light source or a free-electron laser. In one example, the light source may be a broadband light source, such as a white light or supercontinuum light source. In another example, the multi-wavelength light source includes a laser array configured to emit light at one or more wavelengths.

FIG. 1B illustrates another configuration of a sensing system 101. Sensing system 101 is identical to sensing system 100, except that in sensing system 101, light is also propagated in the backward direction 106 due to, for example, Rayleigh scattering 104 towards the proximal end and detected by detector 108 placed at the proximal end. Propagating light in the backward direction may also be possible by a reflective element at or adjacent the distal end, such as depositing a reflective coating at the distal end of optical conduit 102 or placing a retroreflector external to optical conduit 102. FIG. 1C illustrates schematically an example of analyzer 110. Analyzer 110 may include a computer processing system 122. Computer processing system 122 (in the present example) includes at least one processing unit 124 which may be a single computational processing device (e.g. a microprocessor or other computational device) or a plurality of computational processing devices. Processing unit 124 may be configured for tasks such as determining attributes of a selected species in a fluid. Through a communications bus 126, processing unit 124 is in data communication with a system memory 128 (e.g. a read only memory storing a BIOS for basic system operations), volatile memory 130 (e.g. random access memory such as one or more DRAM modules), and non-transient memory 132 (e.g. one or more hard disk drives, solid state drives, flash memory devices and suchlike). Instructions and data to control operation of processing unit 124 are stored on the system, volatile, and/or non-transitory memory 128, 130, and 132. Various databases, as discussed below, may also be stored on memory 128, 130, and 132.

Computer processing system 122 also has one or more input/output interfaces 134 which allow the system 122 to interface with a plurality of input/output devices 136. As will be appreciated, a wide variety of input/output devices may be used, for example keyboards, pointing devices, touch screens, touch-screen displays, displays, microphones, speakers, hard drives, solid state drives, flash memory devices and the like. Computer processing system 122 also has one or more communications interfaces 138, such as Network Interface Cards, allowing for wired or wireless connection to a communications network 140 such as a local or wide area network.

Computer processing system 122 stores in memory and runs one or more applications allowing operators to operate the device system 122. Such applications will typically comprise at least an operating system such as Microsoft Windows, Apple OS X, Unix, Linux or Android.

The description hereinafter refers to the configuration of sensing system 101 relying on detecting backward propagating light due to Rayleigh scattering. It should be appreciated by a skilled person that the described principles are also applicable to sensing system 100, or sensing system 101 relying on detecting backward propagating light due to reflection or retroreflection. Also, hereinafter, an “input end” refers to the end of the optical conduit receiving light as an input and the output end refers to the end of the optical conduit providing light as an output. Accordingly, the input end and output end may be opposite ends of the optical conduit, or the same end of the optical conduit.

Sorptive Portion

Sorptive portion 105 has a set of different sorption properties associated with the multiple selected species. The different sorption properties may each be any one or any combination of an absorption property, an adsorption property and an ion-exchange property. In use, the sorptive portion reversibly sorbs at least one of the multiple selected species to vary an optical characteristic of sorptive portion 105. An advantage of the sorptive portion being reversibly sorptive is that any increase or decrease in concentration of the selected species may be monitored in substantially real time.

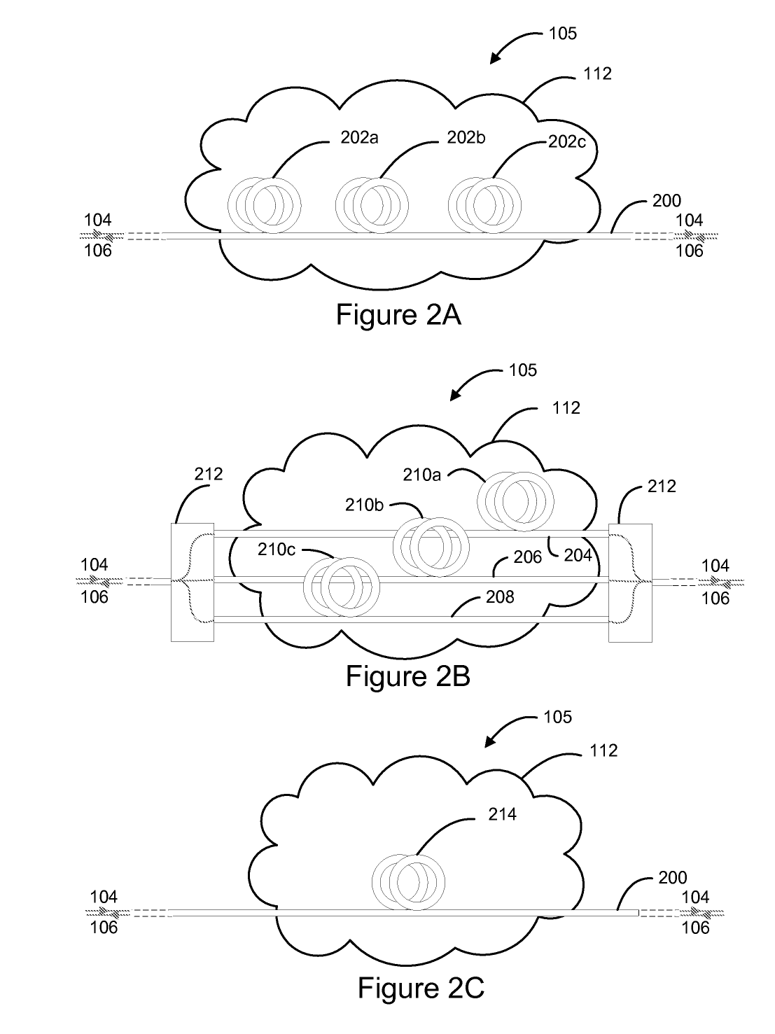

FIGS. 2A to 2C illustrate different arrangements of sorptive portion 105. In FIG. 2A, sorptive portion 105 includes multiple sorptive elements each having a different sorption property with respect to the species for which the sensor is designed. In this arrangement, optical conduit 102 is a single optical fiber 200, where the sorptive elements are in the form of sorptive sections (e.g. 202a, 202b and 202c) of optical fiber 200. The multiple sorptive sections may be closely spaced along optical fiber 200 such that the multiple sorptive sections may be positioned in fluid 112. The multiple sorptive sections may each be in the form of a coil, which improves spatial resolution of the measurement.

The schematic figure shows a separation between sorptive elements 202a, 202b and 202c. This is for clarity of illustration. In practice, the coiled regions may be closely adjacent to one another so that all the elements are exposed to substantially the same fluid.

In FIG. 2B, optical conduit 102 includes a bundle of optical fibers (e.g. 204, 206 and 208), each of which includes a sorptive element (e.g. 210a, 210b and 210c), which may extend along only a section of the respective optical fiber or the entire optical fiber. Similar to those in FIG. 2A, each sorptive element exhibits a distinct sorption property with respect to the species for which the sensor is designed.

In FIG. 2C, sorptive portion 105 includes a single sorptive element 214 exhibiting the set of different sorption properties. The single element may be adapted to sorb more than one of the multiple selected species to vary an optical property of the sorptive portion.

In the arrangements of FIGS. 2A and 2B, the multiple sorptive elements may each be adapted to sorb a different one or more of the multiple selected species to vary an optical property of the sorptive portion. Each sorptive element is therefore used to sense a particular one or more species. In the arrangement of FIG. 2C, single sorptive element 214 is used to sense more than one species.

Each sorptive element may include a reactive component and a host component that co-operate to provide the desired sorptive properties for the sorptive element. The role of the reactive component is to functionalise the host component of the sorptive element, such that the host component is securely adhered to or embedded in the optical conduit while the reactive component reacts (e.g. forms a bond) with a corresponding species. When the reactive component reacts with the corresponding species, an optical characteristic of the sorptive portion (e.g. absorption coefficient, absorption spectrum or dispersive characteristic) can be varied. An example is a paint primer used to provide a key for a fluorescent coating. In this example, the fluorescent coating(s) can be regarded as a reactive component, reacting to external stimuli (e.g. chemicals or UV light) to produce a signal or a variation in an optical characteristic at a certain wavelength or wavelengths. The fluorescent coating(s) can, in some cases, however be difficult to be applied directly to a substrate (e.g. a metal surface) and so a primer may be used to create a stable surface to support the fluorescent material. Doing so functionalises the primer which has adsorption properties to the substrate. Alternatively the fluorescent material may be mixed in with the primer.

Forward propagating light 104 may continue to propagate to another fluid, in which another sorptive portion (e.g. a set of multiple sorptive sections or a single sorptive section) may be positioned, and subsequently detected by detector 108. Further, in the arrangement shown in FIG. 2B, optical conduit 102 includes a 3×1 coupler 212 at each end for splitting and combining forward propagating light 104 and backward propagating light 106.

Hereinafter sensing system 101 is described primarily with reference to the example using the arrangement of FIG. 2A (i.e. multiple sorptive elements in a single optical fiber), but it should be apparent to a skilled person that the description is also applicable to the example using a fiber bundle or examples using other optical waveguides such as a slab waveguide. Further, description directed to a sorptive section of an optical fiber may also be applied more generally to a sorptive element, which includes a sorptive section of an optical fiber or an entire sorptive optical fiber.

Depending on the variation of the optical characteristic to be relied upon, detector 108 may be correspondingly configured to detect a different feature of the light at the output end.

Variation in Light Confinement

In one arrangement, the optical characteristic of the sorptive portion responsive to the sorption of one or more species may include light confinement. In this instance, the detected feature of the light may include optical power. Determination of species attributes using variation in light confinement may be understood as follows.

Optical fiber 200 has a core surrounded by a cladding. The cladding may in turn be surrounded by a coating. Optical fiber 200 is designed such that the refractive index of the core (n_{core}) is higher than that of the cladding (n_{clad}). In a simplified model using geometrical or ray optics, light is understood to be confined in the core by total internal reflection when light rays are incident on the core-cladding interface at an angle greater than the critical angle. In a more realistic model using physical or wave optics, light is understood to be guided in substantial confinement in the core, with a portion of light extending into the cladding as evanescent field. The degree to which the evanescent field is extended into the cladding is a function of n_{core} and n_{clad}. In general, a smaller difference between n_{core} and n_{clad} results in a lesser degree of light confinement in the core and more evanescent field extending into the cladding. In practice, because the cladding is not infinitely thick, the evanescent field further extends into the coating or the space (e.g. air) surrounding optical fiber 200 giving rise to confinement loss as light propagates along optical fiber 200. As a result, any change in the refractive index of the cladding or the coating leads to a change in confinement loss.

As mentioned, optical fiber 200 includes multiple sorptive sections 202a, 202b and 202c. Each sorptive section has a different sorption property associated with one or more of the plurality of selected species. Each sorptive section may be engineered such that one or more selected species is sorbed differentially into a sorptive outer layer, such as an absorptive cladding or an adsorptive coating, of the sorptive portion. Sorption of species into the cladding and/or coating gives rise to a change, such as an increase or a decrease, in their refractive indices. For example, an increase of refractive index in the cladding and/or coating in turn gives rise to an increase in confinement loss as light propagates along optical fiber 200. The description hereinafter refers to an increase of refractive index without loss of generality. A skilled person in the relevant art would appreciate the described arrangements are also applicable to sorptive section where sorption of a species causes a decrease in refractive index.

Furthermore, guided light in optical fiber 200 propagating in the forward direction may experience backscattering, such as Rayleigh scattering 104, in an opposite direction. In sensing system 101, light source 114 is a pulse laser and is configured to regularly launch light pulses into a proximal end of optical fiber 200 in the forward direction 104, whereas detector 108 is configured to receive and detect at the proximal end at least one feature of the backward propagating light 106. The detected feature may contain information as to any change in, or otherwise associated with, confinement loss in the sorptive sections arising from the presence of any one of the plurality of selected species. Analyzer 110 may be configured to determine one or more attributes, such as the presence, or the concentration and ratio, of the plurality of selected species in the fluid based on the detected feature.

In one example, detector 108 is configured to measure the average optical power of the backward propagating light 106 against the time it takes for the backscattered light pulses to return. By measuring the average optical power, which is affected by light confinement loss, the presence and/or the concentration of the plurality of selected species in the fluid may be estimated. By measuring the round-trip time of the light pulses, the round-trip distance travelled by the light pulses (and hence distance d from the proximal end of optical fiber 200) may be estimated by d=ct/(2n_{eff}) where t is the round-trip time, c is the speed of light in vacuum and n_{eff} is the effective refractive index of optical fiber 200.

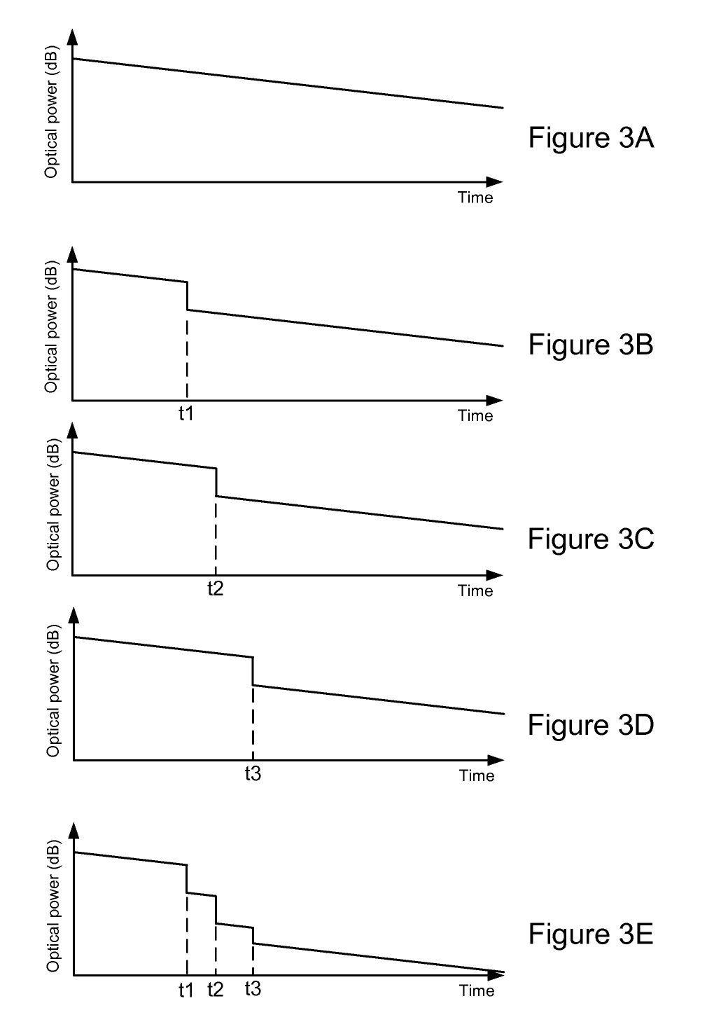

FIG. 3A illustrates an example of the average optical power (in a logarithmic scale) measured by detector 108 over time, which can be readily converted into distance from the proximal end of optical fiber 200 using the above equation. The linear decline (in a logarithmic scale) of the average optical power is characteristic of Rayleigh scattering loss and represents Rayleigh scattering along an optical fiber without any sorptive portion or in the absence of any of the selected species. Since the extent of the linear decline also depends on a number of other factors, such as temperature, pressure and strain experienced by the sorptive portion, the linear decline in FIG. 3A represents a baseline measurement. It is envisaged that optical fiber 200 may include a non-sorptive section for such calibration purposes. In the example using a fiber bundle illustrated in FIG. 2B, the sensing system may include an additional non-sorptive optical fiber for calibration or compensation purposes. Analyzer 110 may include a calibration database for storing the baseline measurement.

FIGS. 3B to 3D illustrate examples of the average optical power (in a logarithmic scale) measured by detector 108 over time, when different species are present in fluid 112. FIGS. 3B to 3D each indicate a single abrupt drop in average optical power over time against a background of linear decline as illustrated in FIG. 3A. The power drops correspond to a confinement loss in each of sorptive section 202a, 202b and 202c, respectively, and indicate presence of one or selected species which the respective sorptive section is responsive to. Because sorptive section 202a is closer to the proximal end of optical fiber 200, FIG. 3B depicts an optical power drop earliest in time at t1. Similarly, because sorptive section 202c is furthest from the proximal end of optical fiber 200, FIG. 3D depicts a power drop latest in time at t3. The round-trip time, and hence the distance from the proximal end of optical fiber 200, at which the power drop occurs may therefore correspond to an indication of the presence of a particular one or more of the selected species. For example, analyzer 110 may include a location database storing the distance (or corresponding round-trip time or the corresponding species expected for sorption) of each sorptive section. Analyzer 110 may be configured to determine whether a power drop is observed at a particular stored distance, in which case analyzer 110 may determine that a particular one or more of the selected species is present in fluid 112.

In practice, even when only one selected species is present in fluid 112, all three sorptive sections 202a, 202b and 202c may have an increased confinement loss. FIG. 3E illustrates another example of the average optical power (in a logarithmic scale) measured by detector 108 over time, when one or more selected species are present in fluid 112. The three abrupt power drops correspond to a confinement loss in sorptive sections 202a, 202b and 202c, respectively.

The absolute extent of a power drop for each of the three sorptive sections may also provide information of concentration of the selected species. In general, a larger power drop indicates a greater confinement loss, which in turns indicates a decreased difference between n_{core} and n_{clad} and hence a greater concentration of the species. The relative extent of a power drop among the three sorptive sections may provide information of the presence of the selected one or more species. FIG. 3E illustrates equal power drops at t1, t2 and t3. This may, as a hypothetical example, indicate the presence of species #1, 2 and 3 of 20 selected species. In another example, if the power drops at t1 are twice as much as the power drops at t2 and t3, this may indicate presence of species #4, 5, 6, 7 and 8 out of the 20 selected species. In yet another example, if the power drop at t1 is half as much as the power drop at t2, which is in turn half as much as the power drop at t3, this may indicate presence of species #9, 10, 11, 12, 13, 14, 15 and 16 of the 20 selected species. The number of selected species detectable may be fewer than or more than 20. In principle, there may be no limit to the number of detectable species provided that the sensing system has the requisite resolution in measuring the absolute and relative power drops. In practice, an increased number of sorptive sections gives rise to an improvement on the resolution and hence increases the number of detectable species.

Accordingly, analyzer 110 may be configured to apply a statistical analysis, such as principal component analysis, to resolve the respective presence and/or concentrations of each selected species. Principal component analysis is a numerical iteration procedure, which begins with an initial guess for the concentrations (including zero to indicate the absence thereof) of each selected species, computes expected power drops and compares with the detected power drops at each of t1, t2 and t3. The procedure may then adjust the concentrations of each selected species, recompute expected power drops and recompare with the detected power drops. The concentrations of each selected species may be re-adjusted at each subsequent iteration. The procedure may terminate when a threshold correlation between the expected power drops and the detected power drops is obtained. In general, a greater number of sorptive elements provides better selectivity of the species present and better sensitivity of the species concentration. Other suitable statistical analysis, such as canonical correlation, may be used.

To improve spatial accuracy or relax the pulse duration requirements, sorptive sections 202a, 202b and 202c may be in the form of a coil. For example, assuming an effective index of 1.5, a light pulse of 1 ns duration has a spatial length of approximately d=ct/(n_{eff})=(3.0×10⁸)×(1×10⁻⁹)/1.5=0.2 m. Therefore, a 2 metre long sorptive section coiled into a space of 0.2 m may relax the pulse duration to approximately 10 ns. Alternatively, coiling in this manner and still using light pulses of 1 ns duration may improve the spatial resolution to approximately 0.02 m.

Variation in Spectroscopic Characteristics

In another arrangement, the optical characteristic of the sorptive portion responsive to the sorption of particular species may include spectroscopic characteristics. In this instance, the at least one feature of the light detected may include spectral information. The optical conduit of FIG. 2C with a single sorptive element may be suited to this arrangement. Determination of species attributes using variation in spectral information may be understood as follows.

Sorption portion 105 may be embedded or coated with a material to exhibit a first spectral response to sorption of a first selected species and a second spectral response to sorption of a second selected species. The spectral response may be a specific transmission spectrum, for example, a power loss at a particular wavelength.

In response to sorption of both the first and the second species, sorption portion 105 may exhibit a spectral response which is a combination of the first and the second responses. In other words, sorption portion 105 may act like a spectral filter in response to sorption of any one of more of the multiple selected species.

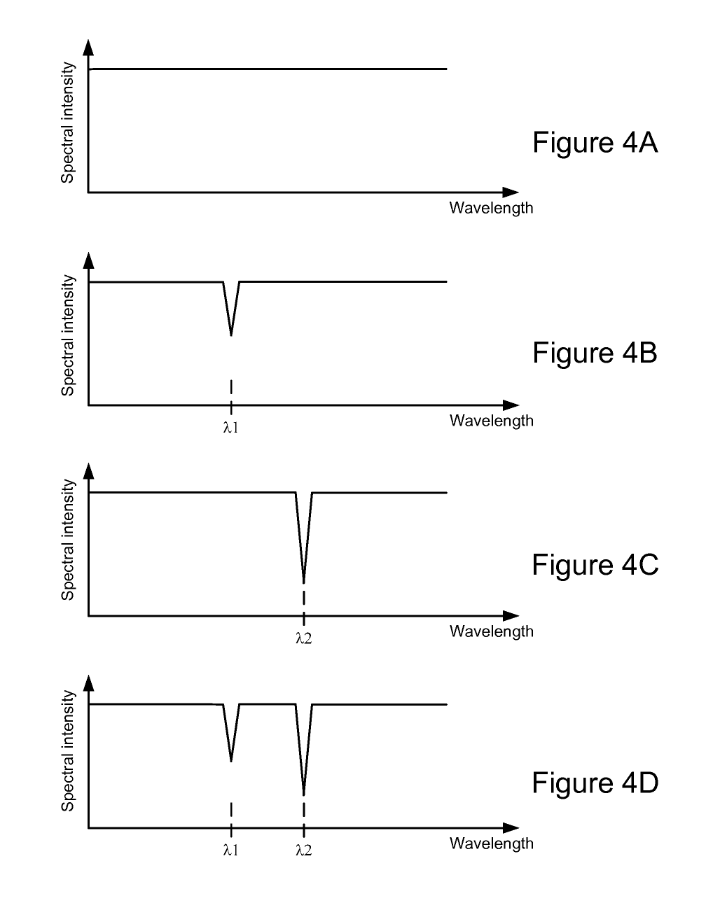

In one arrangement, detector 108 is configured to measure the spectral information, such as spectral content, of the backward propagating light 106. FIG. 4A illustrates an example of the spectral density measured by detector 108 against wavelength in the absence of any selected species. The flat spectrum in FIG. 4A indicates that there is no power loss at any wavelength. Since the flatness of the measured spectrum may also depend on a number of other factors, such as temperature, pressure and strain experienced by the sorptive portion, the flat spectrum in FIG. 4A represents a baseline measurement. It is envisaged that optical fiber 200 may include a non-sorptive section for such calibration purposes. In the example using a fiber bundle illustrated in FIG. 2B, the sensing system may include an additional non-sorptive optical fiber for the calibration purposes. Analyzer 110 may include a calibration database for storing the baseline measurement.

FIGS. 4B and 4C illustrate examples of the spectral density measured by detector 108 against wavelength, when a first selected species and a second selected species, respectively, are separately present in fluid 112. FIGS. 4B and 4C each illustrate a single abrupt drop in spectral density at respective wavelengths λ1 and λ2, corresponding to the spectral responses associated with the first selected species and the second selected species, respectively.

FIG. 4D illustrates an example of the spectral density measured by detector 108 against wavelength, when both the first and second selected species are simultaneously present in fluid 112. FIG. 4D indicates abrupt drops in spectral density at both wavelengths λ1 and λ2, indicating presence of both the first selected species and a second selected species.

Analyzer 110 may include a spectral information database storing the spectral response of each selected species. Analyzer 110 may be configured to determine the spectral response to which a particular selected species is observed, in which case analyzer 110 may determine that the particular one of the selected species is present in fluid 112.

The extent of a spectral power drop may indicate the concentration of the corresponding species. In general, a larger power drop indicates a greater confinement loss, which in turns indicates a decreased difference between n_{core} and n_{clad} and hence a greater concentration of the corresponding species.

In practice, sorptive portion 105 may exhibit multiple spectral responses to sorption of multiple selected species. Furthermore, spectral responses for different species overlap. In this instance, analyzer 110 may be configured to apply principal component analysis to resolve the respective presence and/or concentrations of each selected species. The principal component analysis may begin with an initial guess for the concentration of each selected species, computes an expected spectral response and compares with the detected response. The procedure may then adjust the concentrations of each selected species, recompute an expected spectral response and recompare with the detected response. The concentration of each selected species may be re-adjusted at each iteration, and the procedure may terminate when a threshold correlation between the expected response and the detected response is obtained.

Method

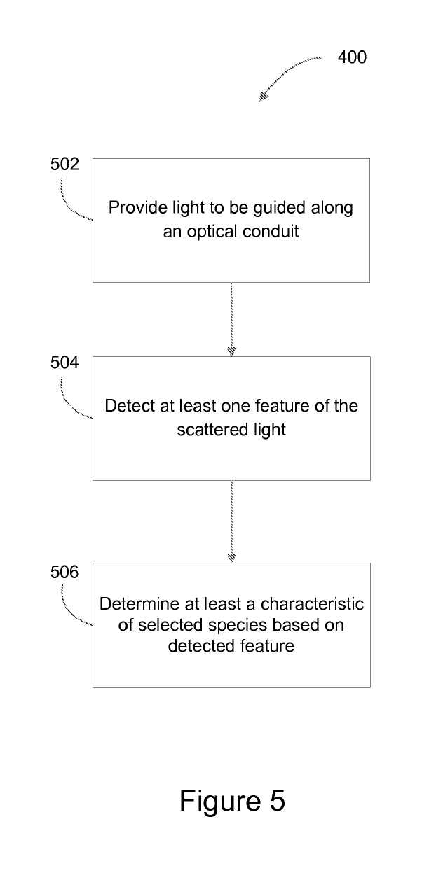

FIG. 5 illustrates a method 500 for operating the described sensing system.

At step 502, light is provided by, for example, light source 104 to be guided in an optical conduit from an input end to an output end. The optical conduit includes a sorptive portion having a set of different sorption properties associated with the multiple selected species. The sorptive portion is adapted to be positioned in the fluid to reversibly sorb at least one of the multiple selected species to vary an optical characteristic of the sorptive portion.

At step 504, for each of the sorptive elements, at least one feature of the light at the output end associated with optical characteristic is detected by, for example, detector 106.

At step 506, at least one attribute of at least one of the multiple selected species in the fluid is determined based on the detected feature by, for example, analyzer 110. In some embodiments, analyzer 110 is configured to determine the presence and/or concentration of the plurality of selected species by principal component analysis.

The step of determining includes determining the presence and/or concentration of the plurality of selected species by principal component analysis.

Emulsions

In an application, the sensing system is used to indicate the relative concentration of two phase-separated or immiscible species, such as water and oil, forming an emulsion. Using a water-oil emulsion as the target sensing example, the sensing system includes an oleophilic sorptive element, which attracts and sorbs oil in the emulsion, and a hydrophilic sorptive element, which attracts and sorbs water in the emulsion. The detector may be configured to detect a power drop or spectral information as discussed above. The power drop or spectral information may be associated with the variation in light confinement or spectroscopic characteristic of the sorptive elements. The analyzer may be configured to determine the relative concentration of water and oil in the emulsion based on the detected power drop or spectral information.

Now that embodiments of the sensing system are described, it should be apparent to the skilled person in the art that the described sensing system has the following advantages:

The sensing system may facilitate monitoring of leakage of harmful or hazardous materials;

The use of optical fibers allows for long-range monitoring, with the range limited by length of the fiber and the propagation loss of the optical fiber;

Real time and continuous measurement of, for example, hydrocarbon-contaminated water and water-contaminated hydrocarbons (liquid or gas phase), as well as for other trace materials in flow streams are possible;

The sensing system is applicable to settings near plant as well as remote unmanned locations such as deep sea infrastructure, or down-hole environments;

The sensing system allows control systems to take executive actions based on measurements. This in turn enables direct control of multi-phase separators in place of indirect level control to achieve the required level of separation performance.

The sensing system allows discharges to be controlled based on similar executive control actions as an integral part of the overall process control system. The impact of this is to simplify the design of separation systems and reduce size and costs as well as optimization of performance (e.g. higher throughput and reduced chemical usage).

The sensing system enables faster response times to allow more compact facilities with reduced hold-up which can be positioned on the sea bed in deep water, reducing field development costs and increase reserves recovery.

It will be understood that the invention disclosed and defined in this specification extends to all alternative combinations of two or more of the individual features mentioned or evident from the text or drawings. For example, optical conduit 102 may include more (e.g. hundreds) or fewer (e.g. down to 1) sorptive elements. Optical conduit 102 may be optical waveguides other than optical fibers or slab waveguides. All of these different combinations constitute various alternative aspects of the invention.

1. A sensing system for sensing multiple selected species in a fluid, the sensing system comprising:
an optical conduit for guiding light from an input end to an output end, the optical conduit comprising a sorptive portion having a set of different sorption properties associated with the multiple selected species, the sorptive portion adapted to be positioned in the fluid to reversibly sorb at least one of the multiple selected species to vary an optical characteristic of the sorptive portion;
a detector for detecting at least one feature of the light at the output end associated with the optical characteristic; and
an analyzer for determining at least one attribute of at least one of the multiple selected species in the fluid based on the detected feature.

2. The sensing system of claim 1 wherein the sorptive portion comprises multiple sorptive elements each exhibiting a subset of the set of different sorption properties.

3. The sensing system of claim 2 wherein the multiple sorptive elements are each adapted to sorb a different one or more of the multiple selected species.

4. The sensing system of claim 2 wherein the multiple sorptive elements are multiple sorptive sections of an optical fiber.

5. The sensing system of claim 2 wherein the multiple sorptive elements are multiple sorptive sections in respective multiple optical fibers.

6. The sensing system of claim 1 wherein the optical characteristic of the sorptive portion comprises light confinement characteristic responsive to sorption of one or more of the multiple selected species.

7. The sensing system of claim 6 wherein the at least one feature of the light detected comprises optical power.

8. The sensing system of claim 1 wherein the optical characteristic of the sorptive portion comprises spectroscopic characteristic responsive to sorption of one or more of the multiple selected species and the at least one feature of the light detected comprises spectral information.

9. The sensing system of claim 2 wherein at least one of the multiple sorptive elements includes a reactive component and a host component that co-operate to provide a desired sorption property of the sorptive element.

10. The sensing system of claim 1 wherein each of the different sorption properties is selected from a group consisting of: an absorption property, an adsorption property and an ion-exchange property.

11. The sensing system of claim 2 wherein each sorptive element comprises a sorptive outer layer.

12. The sensing system of claim 11 wherein the sorptive outer layer comprises an absorptive cladding layer of an optical fiber.

13. The sensing system of claim 11 wherein the sorptive outer layer comprises an adsorptive coating layer of an optical fiber.

14. The sensing system of claim 1 wherein the optical conduit comprises a non-sorptive element for calibration.

15. The sensing system of claim 1 wherein the input end and the output end are opposite ends of the optical conduit.

16. The sensing system of claim 1 wherein the input end and output end is the same end of the optical conduit.

17. The sensing system of claim 1 wherein the light source comprises a pulsed light source.

18. The sensing system of claim 17 wherein the pulsed light source comprises a pulsed laser.

19. The sensing system of claim 17 wherein the light source comprises a multi-wavelength light source.

20. A method for operating a sensing system for sensing multiple selected species in a fluid, the method comprising:
providing light to be guided in an optical conduit from an input end to an output end, the optical conduit comprising a sorptive portion having a set of different sorption properties associated with the multiple selected species, the sorptive portion adapted to be positioned in the fluid to reversibly sorb at least one of the multiple selected species to vary an optical characteristic of the sorptive portion;
detecting at least one feature of the light at the output end associated with the optical characteristic; and
determining at least one attribute of at least one of the multiple selected species in the fluid based on the detected feature.

21. The method of claim 20 wherein the step of determining at least one attribute comprises determining the presence and/or concentration of the plurality of selected species.

22. The method of claim 21 wherein the step of determining the presence and/or concentration of the plurality of selected species comprises performing a statistical analysis of the detected features.

23. The method of claim 22 wherein the step of performing a statistical analysis comprises performing a principal component analysis.
